(19) United States
(12) Patent Application Publication
(10) Pub. No.: US 2024/0149013 A1
(43) Pub. Date: May 9, 2024

(54) PAIN TREATMENT SYSTEM

(71) Applicant: Joseph MANNE, New York, NY (US)

(72) Inventor: Joseph MANNE, New York, NY (US)

(21) Appl. No.: 18/505,304

(22) Filed: Nov. 9, 2023

Related U.S. Application Data
(60) Provisional application No. 63/382,915, filed on Nov. 9, 2022.

(51) Int. Cl. A61M 21/02; G16H 10/60

(52) U.S. Cl. CPC A61M 21/02; G16H 10/60; A61M 2021/0016

(57) ABSTRACT

A method, system and apparatus for treatment of pain which stimulates one or more of the five senses to reduce pain in a human. The five senses being visual (eyes), auditory (ears), olfactory (smell), gustatory (taste), and tactile (touch). The apparatus of the invention preferably uses a headset having an audio and video system along with a scent delivery device, food/tastants, and a haptic device to stimulate one or more of the five senses of the human.

### CROSS-REFERENCE TO RELATED APPLICATIONS

This application claims the benefit of U.S. Provisional Application No. 63/382,915 filed on Nov. 9, 2022 the contents of which is incorporated herein by reference.

### BACKGROUND OF THE INVENTION

This invention relates to reducing pain in a human and more particularly to a method, system and apparatus for stimulating one or more of the senses to reduce pain in the human.

### PRIOR ART

Stimulating the senses of a human to reduce stress, anxiety and stress related disorders without the use of pharmacological treatments is known. Generally, audio, visual and aromatic stimulations have been used to reduce stress and anxiety. There is still, however, a need for reducing pain in human, especially without the use of or as an enhancement to pharmacological treatments for reducing pain.

### SUMMARY OF THE INVENTION

A method, system and apparatus for treatment of pain has been discovered which stimulates one or more of the five senses to reduce pain in a human. The five senses being visual (eyes), auditory (ears), olfactory (smell), gustatory (taste), and tactile (touch). The apparatus of the invention preferably uses a headset having an audio and video system along with a scent delivery device, food/tastants, and a haptic device to stimulate one or more of the five senses of the human.

In the present invention, a database is formed having one or more recordings for each of the five senses. Preferably, the database has a plurality of recordings for each of the five senses. In the database, there are five categories of recordings, each category corresponding to one of the five senses. More preferably, each one of the recordings in each of the categories is associated with one of the recordings in each of the other categories in the database. This association allows the selection of subsequent recording based a selection of an initial recording.

One or more of the recordings is selected from the database for treating the human who has a level of pain. Preferably, from the database, three or more of the recordings are selected. Of the selected recordings, it is preferred that at least one visual, one auditory, and one olfactory recording are selected. Gustatory and tactile recordings can also be optionally selected.

The selected recordings are administered to a patient who is in pain. Preferably, the selected recordings are administered to the human simultaneously such that a plurality of the senses of the human are stimulated at the same time. Such a simultaneous stimulation of the senses, as opposed to sequential individual stimulation, is deemed to have a superior pain reduction effect on a human.

During the administration of the selected recordings, the level of pain in the patient is monitored to determine if there is a decrease in the pain. If there is a decrease the pain, then the selected combination of recordings is continued to be administered until such time as the pain returns to its initial level.

If there is no decrease in pain during the administration of the selected recordings, then a new selection of recordings is made and the new selection of recording is administered to the patient. Again, the level of pain in the patient is monitored during the administration of the newly selected recordings to determine if there is a decrease in pain. If there is a decrease in pain, then the newly selected recordings are continued to be administered until the pain returns to its initial level. If the newly selected recordings do not decrease the pain, then a different selection of recordings is made, and the process is repeated. This process is continued until the selection recordings decreases the pain. Once a set of selected recordings is found to decrease the pain, that set of recordings is continued to be administered to the patient until such time as the pain returns to its initial level.

The database also preferably stories record of a patient. The records of the patient stored in the database can include collected information from the patient. The collected information from the patient can include the age of the patient, coexisting diseases of the patient, the gender of the patient, prior treatments for pain in the patient, the current level of pain in the patient, the duration of the pain, the anatomical location of the pain in the patient, the current pain management of the patient, current medications being used by the patient, and preferred sensory treatments for pain in the patient. Furthermore, the selected recordings that decrease the pain of the patient can be stored in the database for that patient, so they can be used again or so they can be used with a different patient who has similar characteristics to the patient. The selected recordings that do not reduce the pain can also be stored in the database, so they are not selected again.

The selection of the recordings can be (1) random, (2) based on information collected from the patient, (3) based on the first selected recording, (4) based on information collected from a previous patient and the selected recordings that reduce the level of pain in the previous patient. The selection can be based on the first selected recording because it is preferred that each of the recordings in each of the categories is associated with one of the recordings in another category. This association can be relied on for the selection of all of the recordings to be administered to the human after the first recording is selected. The database is searchable such that selected recordings that reduced the level of pain in the previous patient can be located and used on a current patient when there are similarities in the collected information of the previous patient and the current patient. In other words, the patient records in the database can be searched to locate previous patients who have similar characteristics to a current patient.

The pain measurements are made using standard pain scales such as VAS and Wong Baker.

The present invention can treat pain that is classified as acute, chronic, and acute on chronic. The pain can be further categorized as neuropathic or somatic.

Monitoring the level of pain and the decrease in pain during the administration of the recordings is recorded in the database along with the duration that the recordings decrease the pain.

In the present invention, the method, system and apparatus for reducing pain in a human having a level of pain has been discovered which preferably stimulates at least three or more of the senses of a human and reduces pain level of the human during the stimulation.

In general, the method can be describe as comprising:
a) providing a database having a plurality of individual visual recordings, auditory recordings, and olfactory recordings;
b) selecting from the database one visual recording, one auditory recording, and one olfactory recording;
c) forming a combination of the selected visual recording, selected auditory recording and selected olfactory recording;
d) delivering the combination to the human having a level of pain;
e) measuring the level of pain in the human during the delivery of the combination to the human;
f) determining if the combination reduced the level of pain in the human;
g) continuing to deliver the combination to the human, if it was determined that the combination reduced the level of pain in the human until the level of pain returns to an initial level; and
h) repeating steps b) through f) with a different combination, if it was determined that the combination did not reduce the level of pain in the human, until a different combination is determined to reduce the level of pain in the human, and then continuing to deliver the different combination that reduced the level of pain in the human until the level of pain returns to the initial level.

The method can include collecting information from the human, the information including age, coexisting diseases, gender, prior treatments for pain, level of pain, duration of pain, anatomical location of the patient's pain, current pain management, specific medications used in the past, current medications being used, and preferred sensory treatment for pain. The selection step b) can be based on the collected information. The method can also include storing the collected information in the database and a record of the patient.

The method can include in the selection step b), the visual recording being selected first, the auditory recording being selected second based on the selected visual recording, and the olfactory recording being selected third based on the selected visual recording and selected auditory recording.

The method preferably includes the database having a plurality of individual gustatory recordings. The selection step b) can include selecting an individual gustatory recording. The formation step c) can include adding the selected gustatory recording to the combination.

Preferably, the method includes, in the selection step b), the visual recording being selected first, the auditory recording being selected second based on the selected visual recording, the olfactory recording being selected third based on the selected visual recording and selected auditory recording, and the gustatory recording being selected fourth based on the selected visual recording, selected auditory recording, and the selected olfactory recording. In this cascading selection of recordings, each selection of recordings after the selection of the initial recording is based on the association of the recording in one category with a recording in the other categories.

Preferably, the method includes the database having a plurality of individual tactile recordings; the selection step b) includes selecting an individual tactile recording; and the formation step c) includes adding the selected tactile recording to the combination.

Preferably, the method includes, in the selection step b), the visual recording is selected first, the auditory recording is selected second based on the selected visual recording, the olfactory recording is selected third based on the selected visual recording and selected auditory recording, the olfactory recording is selected fourth based on the selected visual recording, selected auditory recording, and the selected olfactory recording; and the tactile recording is selected fifth based on the selected visual recording, selected auditory recording, and the selected olfactory recording.

The method of the present invention for treating pain can comprise the steps of: delivering, by a delivery device, a first plurality of combinations of recordings to a patient; determining, by a processor, for a particular combination among the first plurality of combinations, an effect of the particular combination on a pain level of the patient and a duration of the effect of the particular combination on the pain level of the patient, wherein determining an effect of the particular combination on a pain level of the patient and a duration of the effect of the particular combination on the pain level of the patient comprises: determining, by the processor, a measurement of a relationship between a reduction in pain level by the patient while the patient is exposed to the particular combination, and time; determining a particular time representing a moment when the pain level of the patient is reduced; and determining the duration of the effect of the particular combination based on the particular time; generating, by the processor, a delivery schedule for delivery of the particular combination based on the effect and the duration; and delivering, by the delivery device, the particular combination in accordance with the delivery schedule to produce the effect on the pain level of the patient.

### BRIEF DESCRIPTION OF THE DRAWINGS

FIG. 1 illustrates the present invention;

FIG. 2 illustrates scent delivery device for use in the present invention;

FIG. 3 illustrates an audio, visual, gustatory and tactile delivery system for use in the present invention;

FIGS. 4A and 4B illustrates a database for use in the present invention; and

FIG. 5 illustrates another scent delivery device for use in the present invention.

### DETAILED DESCRIPTION OF THE INVENTION

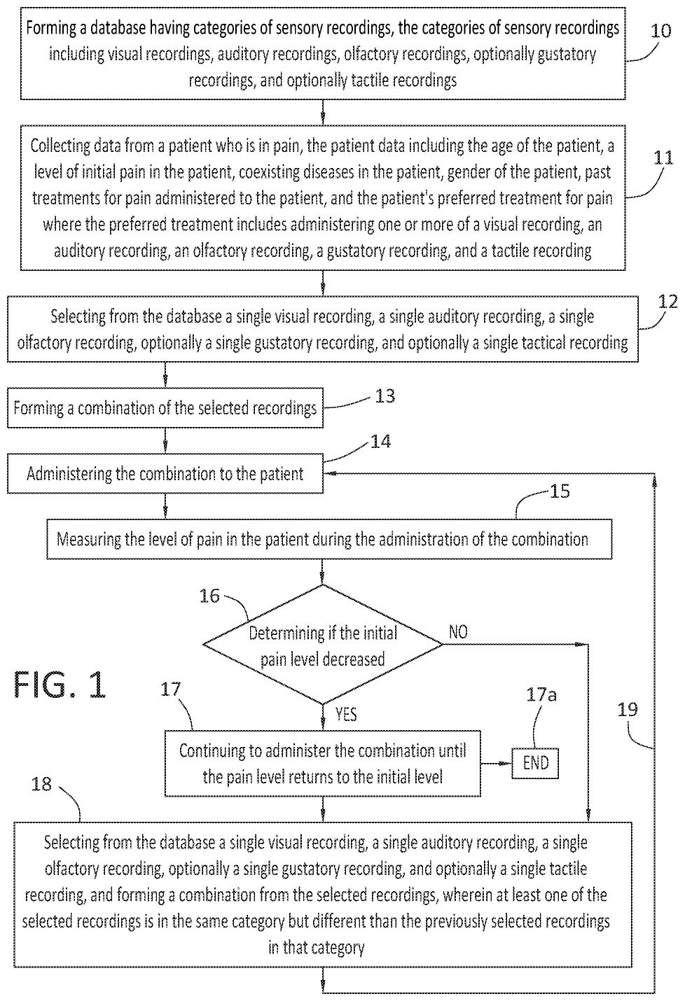

FIG. 1 illustrates the present invention. In step 10 a database is formed having categories of sensory recordings. The database is formed in a conventional manner using conventional equipment, such as a computer with memory. The categories of sensory recordings include visual recordings, auditory recordings, olfactory recordings, optionally gustatory recordings and optionally tactile recordings. Under each category there are a plurality of individual recordings. Preferably, each individual recording in each category is associated with one of the individual recordings in each of the other categories. This association allows for the selection of an individual recording in another category based on a selection of an individual recording in a previous category. In other words, if a picture or video recording is selected under the visual category, the selected picture or video recording is associated with an audio recording in the auditory category such that once the picture or video recording is selected the associated audio recording is automatically selected.

In step 11 data is collected from the patient that is in pain. Data can be collected in any conventional manner using conventional equipment such as a questionnaire form that is given to the patient prior to start of treatment. The data that is collected includes age of the patient, a level of initial pain in the patient, coexisting diseases in the patient, gender of the patient, past treatments for pain which were administered to the patient, duration of pain in the patient, anatomical location of pain in the patient, current pain management, previous medications used by the patient, current medications used by the patient, the patient's preferred treatment for pain with the preferred treatment includes stimulating one or more of the five senses (visual, auditory, olfactory, gustatory, and touch) by administering one or more of a visual recording, and auditory recording, and olfactory recording, a gustatory recording, and a tactile recording. This collected information is stored in the database in the records of the patient.

In step 12 a single visual recording, a single auditory recording, and a single olfactory recording are selected from the database. Optionally, a single gustatory recording and optionally a single tactical recording can be selected from the database. In one embodiment of the present invention once the single visual recording selected, the auditory recording is selected based the association between the visual recording in the auditory recording. Then, the olfactory recording is selected based on its association with the selected auditory recording. Optionally, a gustatory recording can be selected, and the selection of the gustatory recording is based on its association with the selected olfactory recording. Additionally, optionally, a tactile recording can be selected, and the tactile recording is selected based on its association with the selected gustatory recording.

In step 13 all of the selected recordings are combined so that they can be delivered to the patient simultaneously.

In step 14 the combined recordings are administered to the patient.

In step 15 level of pain of the patient is measured during administration of the combination of selected recordings.

In step 16 determination is made whether the initial pain level has decreased. If the answer in step 16 is YES, that the pain level has decreased, then the present invention proceeds to step 17 where the combination is continued to be administered to the patient until such time as the pain level returns to its initial level with treatment is stopped or until a decision is reached to stop the treatment.

If the answer is NO in step 16, the initial pain level has not decreased, then the present invention proceeds to step 18 wherein one of the recordings in the administered combination is changed to form a new combination and the new combination of recordings is administered following step 19. Repeating of steps 14, 15, 16, 18, and 19 is continued when the answer in step 16 is NO. Once the answer in step 16 is YES, the present invention moves to step 17 and the end 17a. In changing one of the recordings in the administered combination, only one of the recordings in one of the categories is changed in the same category to form the new combination.

The results of the treatment are added to the records of the patient in the database at end 17a or at an appropriate time before end 17a. In other words, the combination of sensory recordings that reduce the level of pain in the patient are stored in the database in the records of the patient. Additionally, the collected information of the patient can be stored in combination with the combination of sensory recordings that reduce the level of pain in the patient. This increases the searchability of the database.

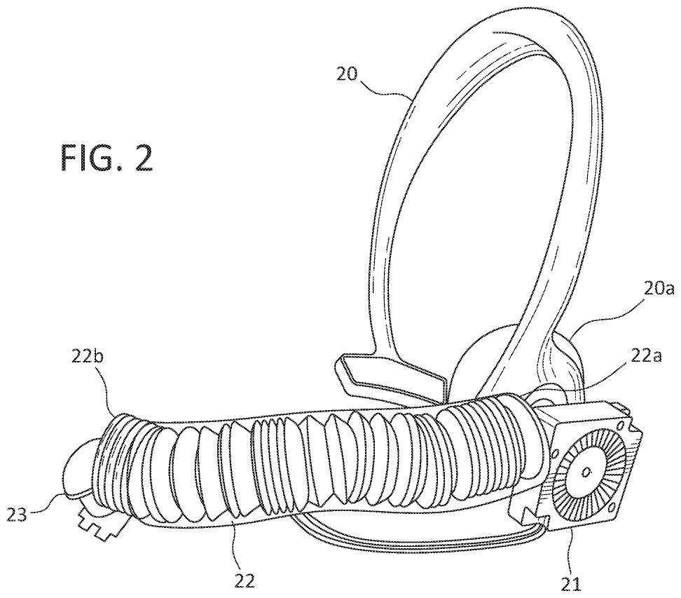

FIG. 2 illustrates a scent delivery device for use in the present invention. The scent delivery device in FIG. 2 has head set 20 with speaker/earphones 20a and an attached blower fan 21 with nozzle 22 extending from the outlet of blower fan 22. One end 22a of nozzle 22 is attached to the outlet of blower fan 21. The other end 22b of nozzle 22 is attached to container 23 containing an aroma component. Nozzle 22 can be a flexible boom tube which allows for adjustment of position of end 22b of nozzle 22 and container 23. Container 23 is positioned in front of the face of the user opposite the nose. Blower fan 21 can be a mini blower fan having the size of 30 mm×30 mm×10 mm. For example, a fan “Delta BFB0312HA” can be used with maximum flow rate of 1.45 cfm in combination with 12-volt DC source and drawing 0.09 amps power. Container 23 can be a 15 ml vial from LA containers. The fan speed can be adjusted with a potentiometer (for example, 10K ohms) to vary the flow rate from about 0 cfm to 1.45 cfm. The intensity of the scent may be inversely related to the flow rate.

To stimulate the sense of taste, gustation, the headset can include a tastant supplier to supply a tastant separately from the scent. For example, the tastant supplier includes a tastant container and a nozzle for delivering a solution of the tastant. The solution of the tastant can be sprayed from the nozzle to the mouth of the patients or directly supplied to the mouth of the patient. The nozzle for tastant is separate from the nozzle for scent.

The fan is used in association with the scent delivery device to create a current of air directed towards the nose or face of the user and thereby assist in the movement of the aroma component to the nose. Suitable fans include blowers, centrifugal or axle fans. Such fans are conventional fans which are small in size. One such fan measures 1½ inches×1½ inches by 1 inch (4×4×2.5 cm) and moves 7 cubic feet of air per minute.

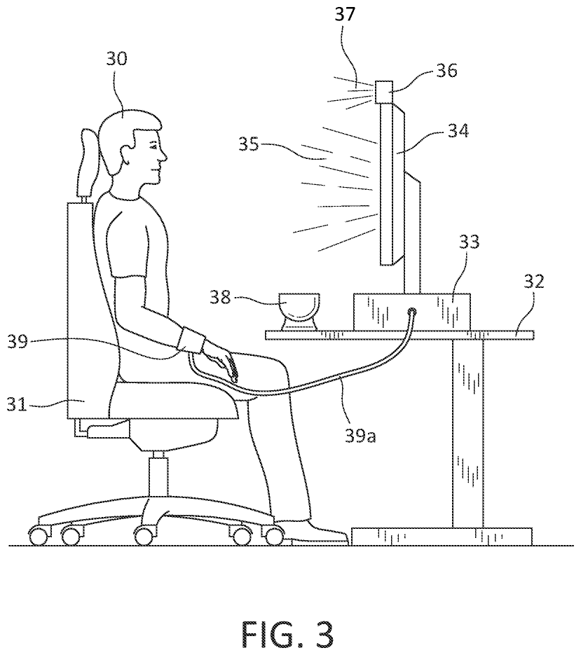

A heater can also be used to assist the movement of scented air to the nose of the patient. The heater can be selected from conventional heaters which are small in size. Heater can heat the air flow from the fan and/or a container of an aroma component to deliver the scent. FIG. 3 illustrates a sensory stimulating system suitable for the present invention. FIG. 3 illustrates patient 30 sitting in chair 31 in front of table 32. On desk 32 is computer 33 having screen 34 which provides image 35 to patient 30 for stimulating the visual senses. Mounted on screen 34 is speaker 36 which provides sound 37 to patient 30 for stimulating the auditory senses. A conventional virtual reality headset can be used in the present invention in place of screen 34 and speaker 36.

Bowl 38 sits on table 32 and holds food that can be eaten by patient 30 to stimulate the gustatory senses.

A conventional tactile interface device is illustrated in FIG. 3 as arm band 39 on the arm of patient 30. Arm band 39 is connected by wire 39a to computer 33 and provides tactile stimulation to patient 30 to stimulate the tactile senses. Conventional tactile stimulations devices can also be used in the present invention.

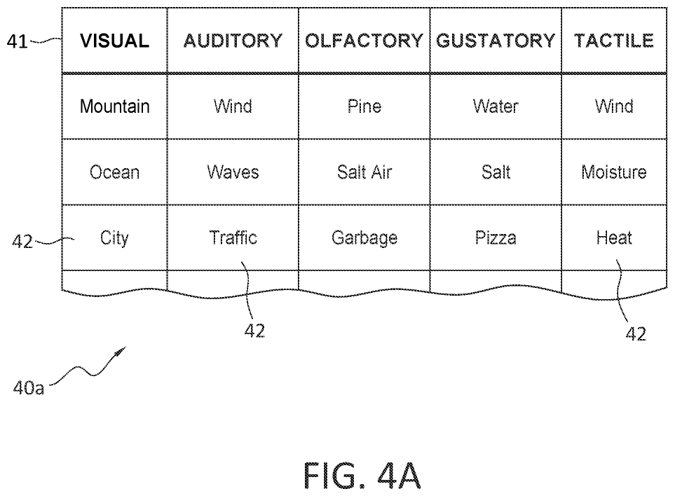

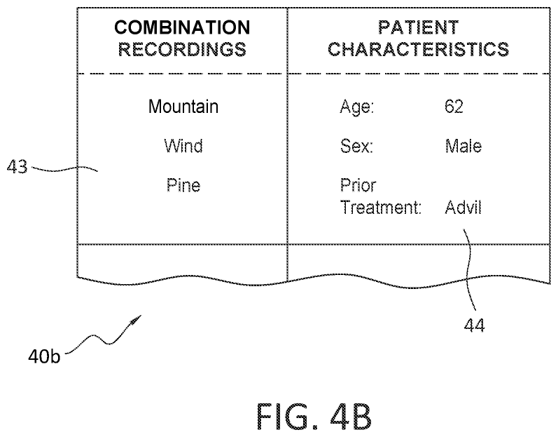

FIG. 4A illustrates part of database 40a for use in the present invention. Database 40 has categories 41 for each of the five senses, visual, auditory, olfactory, gustatory and tactile. In each category 41 there are a plurality of recordings 42. FIG. 4B illustrates part of database 40b where a combination of recordings 43 that reduced pain in the previous patient are stored with characteristics 44 of the previous patient. Characteristics 44 of the previous patient were obtained from collected information of the previous patient. Combination 43 can also be stored in the database in the records of the patient.

Suitably, the data collected from the patients before treatment, during treatment and after treatment as well as the results of the treatments on the patients is stored in the database. The database can be searched for patients having the same or similar characteristics so that the treatment that reduce the pain for one patient can be tried on another patient to see if it will reduce the pain on that other patient. There are patient records in the database. The collected information is stored in the patient records in the database. Also, the sensory recordings used on a patient are stored in the database as part of the patient records in the database. The collected information of the patient can also be appended to the combination of sensory recordings that reduce the pain in the patient.

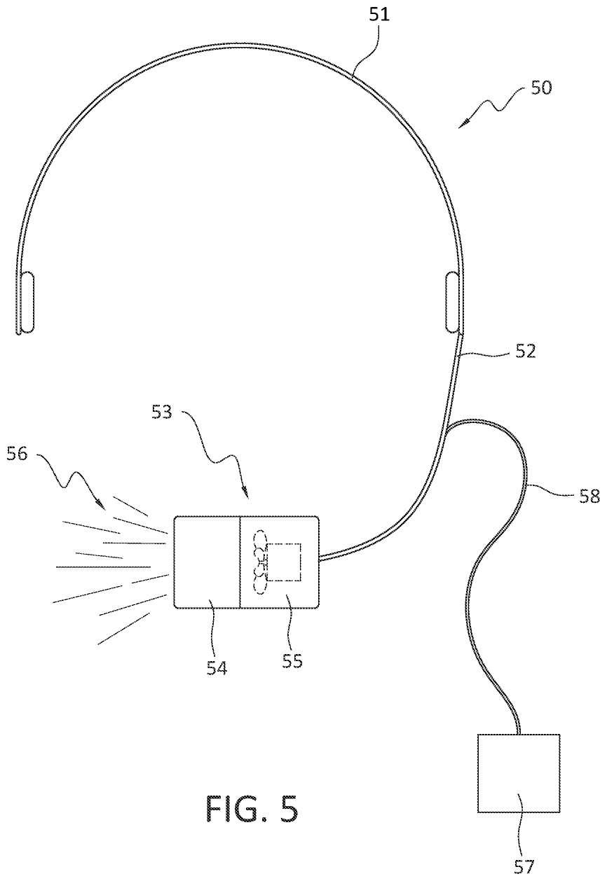

FIG. 5 illustrates a scent delivery device in accordance with the present invention. Scent delivery device 50 has a headset 51 with a flexible arm 52 extending outward from the headset 51. Case 53 is attached at one end of arm 52. In case 53 is a scent container 54 and a fan 55. Scent container 54 is removable from case 53 thereby allowing different scents to be used. Fan 55 moves air through scent container 54 to allow scented air 56 to exit from the case 53. Flexible arm 52 allows for positioning case 53 and directing scented air 56 to the nose of the patient. Electrical source 57 is connected by wire 58 to fan 55 to power fan 55. Headset 51 can be worn by a patient on the head of the patient while power source 57 can be placed in a shirt pocket or set on a table next to the patient.

What is claimed:

1. A method of reducing pain in a human having a level of pain, comprising:
a) providing a database having a plurality of individual visual recordings, auditory recordings, and olfactory recordings;
b) selecting from the database an individual visual recording, auditory recording, and olfactory recordings;
c) forming a combination of the selected visual recording, auditory recording and olfactory recordings;
d) delivering the combination to the human having a level of pain;
e) measuring the level of pain in the human during the delivery of the combination to the human;
f) determining if the combination reduced the level of pain in the human;
g) continuing to deliver the combination to the human, if it was determined that the combination reduced the level of pain in the human until the level of pain returns to an initial level; and
h) repeating steps b) through f) with a different combination, if it was determined that the combination did not reduce the level of pain in the human, until a different combination is determined to reduce the level of pain in the human, and then continuing to deliver the different combination that reduced the level of pain in the human until the level of pain returns to the initial level.

2. The method of claim 1, further comprising:
collecting information from the human, information including age, coexisting diseases, gender, prior treatments for pain, level of pain, duration pain, anatomical location of pain, current pain management, current medications being used, and preferred sensory treatment for pain; and
the selection step b) is based on the collected information.

3. The method of claim 1, wherein:
in the selection step b), the visual recording is selected first, the auditory recording is selected second based on the selected visual recording, and the olfactory recording is selected third based on the selected visual recording and selected auditory recording.

4. The method of claim 1 wherein;
the database further comprises a plurality of individual gustatory recordings;
the selection step b) includes selecting an individual gustatory recording; and
the formation step c) includes adding the selected gustatory recording to the combination.

5. The method of claim 4, wherein:
in the selection step b), the visual recording is selected first, the auditory recording is selected second based on the selected visual recording, the olfactory recording is selected third based on the selected visual recording and selected auditory recording, and the gustatory recording is selected fourth based on the selected visual recording, selected auditory recording, and the selected olfactory recording.

6. The method of claim 1, wherein:
the database further comprises a plurality of individual tactile recordings;
the selection step b) includes selecting an individual tactile recording; and
the formation step c) includes adding the selected tactile recording to the combination.

7. The method of claim 4, wherein:
the database further comprises a plurality of individual tactile recordings;
the selection step b) includes selecting an individual tactile recording; and
the formation step c) includes adding the selected tactile recording to the combination.

8. The method of claim 7, wherein:
in the selection step b), the visual recording is selected first, the auditory recording is selected second based on the selected visual recording, the olfactory recording is selected third based on the selected visual recording and selected auditory recording, the olfactory recording is selected fourth based on the selected visual recording, selected auditory recording, and the selected olfactory recording; and the tactile recording is selected fifth based on the selected visual recording, selected auditory recording, and the selected olfactory recording.

9. The method of claim 2, wherein:
the collected information for one patient is stored in the database and the combination of recordings that reduce the pain in the one patient is stored in database; and
the selection step b) for another patient, includes searching the database for patients to identify one patient having similar characteristics to the other patient and selecting the combination of recordings that reduced pain in the one patient for the selected combination recordings for the other patient.

10. The method of claim 2, wherein:
the collected information of the patient is stored in the database; and
the combination of recordings that reduce the pain in the patient are stored in the database.

11. The method of claim 10, wherein:
the database includes records of the patient; and
the collected information of the patient and the combination of recordings that reduce the level of pain in the patient are stored in the records of the patient in the database.

12. The method of claim 10, wherein:
the database includes records of the patient;
the combination of recordings that reduce the pain in the patient are stored separately from the records of the patient; and
the collected information of the patient is appended to the combination of recordings that reduce the level of pain in the patient that are stored separate from the records of the patient.
